(19) United States
(12) Patent Application Publication
(10) Pub. No.: US 2019/0344095 A1
(43) Pub. Date: Nov. 14, 2019

(54) Light Delivery Apparatus with Optical Comb

(71) Applicant: Medical Coherence LLC, Fairfield, NJ (US)

(72) Inventors: Alon H. Landa, Brookline, MA (US); Eytan Malits, Kiryat Tiv'on (IL)

(21) Appl. No.: 16/407,995

(22) Filed: May 9, 2019

Related U.S. Application Data
(60) Provisional application No. 62/669,601, filed on May 10, 2018.

(51) Int. Cl. A61N 5/06; A61D 99/00; A45D 24/10

(52) U.S. Cl. CPC A61N 5/0616; A61D 99/00; A45D 24/10; A61N 2005/067; A61N 2005/0632; A61N 2005/0644; A61N 2005/0652; A61N 2005/063

(57) ABSTRACT

A light delivery apparatus includes a housing, an optical comb coupled to the housing, and at least one light source inside the housing. The optical comb includes a platform and an array of solid light guides extending from the platform. The optical comb includes an input surface and an exit surface. Substantially all light emitted by the light guides exits from the exit surfaces when in operation as provided by a reflective coating on each of the four or more light guides, as provided by a geometric shape of each of the four or more light guides, or both. The at least one light source is positioned to irradiate the input surfaces when the at least one light source is energized. Also, the at least one light source includes at least one light-emitting diode (LED) unit or at least one laser diode.

### CROSS REFERENCE TO RELATED APPLICATIONS

This application claims the benefit of priority under 35 USC § 119 to U.S. Provisional Patent Application No. 62/669,601, filed on May 10, 2018 and entitled “Low Level Laser Therapy (LLLT) Optical Comb,” which is incorporated herein by reference.

### BACKGROUND

U.S. Pat. No. 9,946,082 (herein “Gerlitz”), filed Dec. 9, 2015 and entitled “Handheld, Low-Level Laser Apparatuses and Methods for Low-Level Laser Beam Production,” is incorporated herein by reference for its pertinent and supportive teachings.

Light therapy is the process of radiating specific wavelengths of light onto the body to stimulate cells, facilitating wound healing and inflammation reduction. Light therapy may be contrasted with other light treatments, such as those designed for hair growth. As the term is used herein, “light” refers to all wavelengths of electromagnetic radiation, such as infrared radiation, and is not limited to the spectrum visible by humans. Directly irradiating targeted inflamed or injured tissue yields the best results. However, many non-treatable objects may absorb or reflect the light between the light source and target tissue, limiting the amount of light that reaches the target tissue. The target tissue requires a threshold of radiation in order to stimulate healing and reduce inflammation. As a result, absorption or reflection of light intended for treatment leads either to no treatment or to very long treatment times as the amount of light that reaches the target tissue may represent a small fraction of the intended light.

The apparatuses and methods herein address the most frequent non-treatable objects that fall between the light source and the target tissue, namely, fur or hair. Light incident on fur is often absorbed or reflected, leading to less than 20% of the incident light passing the fur. Light that passes fur may then interact with other non-treatable material, leading to even less light reaching the target tissue.

### BRIEF DESCRIPTION OF THE DRAWINGS

Some embodiments are described below with reference to the following accompanying drawings.

FIGS. 1-3 show one example of an optical comb in isometric, top, and bottom views, respectively.

FIG. 4 shows a side view of one example of a light therapy apparatus with select internal components represented with dashed lines.

FIG. 5 shows a perspective view of another example of a light therapy apparatus with part of the housing removed and with another example of an optical comb.

FIG. 6 shows a perspective sectional view of the FIG. 5 light therapy apparatus with the housing intact.

FIG. 7 shows a perspective sectional view of select components from the FIG. 5 light therapy apparatus.

### DETAILED DESCRIPTION

The apparatuses and methods herein use an “optical comb” that addresses many of the challenges of traversing the fur in light therapy for animals and delivers more of the light source's power to the skin. The various optical combs herein may also address the fact that an animal environment may be dirty and dirt particles could attach to the comb and affect treatment as well. The optical comb may also be used to traverse hair in humans.

The term “comb” refers more to the optical comb's appearance than to its mode of use. The optical comb will most often operate in a stationary mode for spot treatment, rather than in a combing motion. In this manner, treatment time for an identified area may be noted or tracked.

An optical comb may include multiple light guides configured to direct light received from a light source through the light guides to an exit surface. A light delivery apparatus, such as a light therapy apparatus, including a low-level laser therapy (LLLT) device, includes a light source and may be coupled to the optical comb. The optical comb may be positioned to receive light from the light source. As such, the optical comb guides light from the light source through fur or hair, so the light reaches the skin.

The optical comb delivers light from the light source (minus losses in the apparatus), thereby reducing absorption and reflection from hair or fur as the light guides traverse the hair or fur. The optical comb may be useful for other purposes to traverse a medium through which light delivery is needed. The comb may be coated to increase transmission efficiency through the light guides to the treatment surface. The coating may be an internally reflective coating. The comb may be coated to increase the efficiency of cleaning residue accumulated from oily and dirty hair. A single coating may accomplish both purposes. The distance between light guides may be selected to facilitate close to homogeneous power distribution across the target tissue.

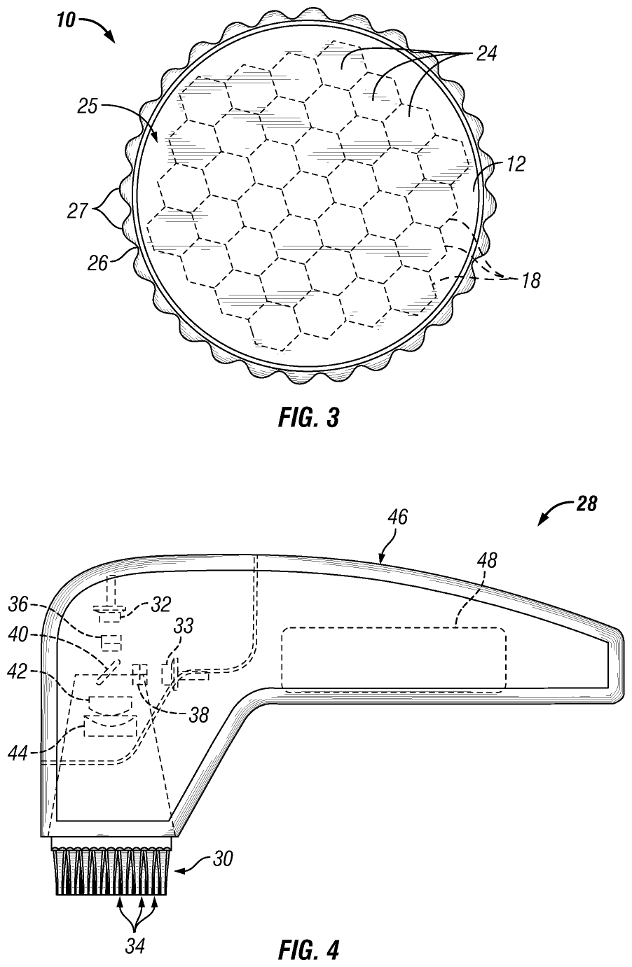

FIG. 4 shows a conceptual side view of one example of a light therapy apparatus 28 with at least one laser diode inside a housing. The features implemented in the FIG. 4 example apparatus may also be implemented in other apparatuses herein. FIG. 4 includes selected internal components useful in understanding a method for operating apparatus 28. Additional components known to skilled persons may be included in a complete apparatus, as may be appreciated from U.S. Pat. No. 9,946,082 to Gerlitz and other references.

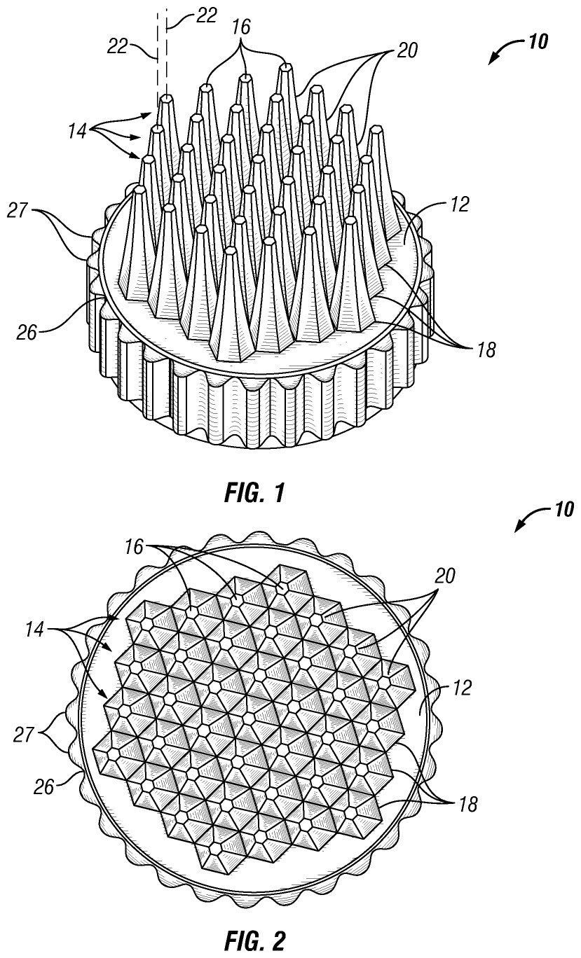

Apparatus 28 includes a detachable optical comb 30, which may be the same as an optical comb 10 shown in FIGS. 1-3 or a modification thereof. Optical comb 30 includes an array of light guides 34 that direct light received from a light source through light guides 34 to a surface outside a housing 46 containing the light source. The outside surface may be tissue targeted for treatment or another type of surface desired for light irradiation.

As light sources, apparatus 28 includes laser diode unit 32 and laser diode unit 33, which provide different wavelengths of light, as discussed in Gerlitz. Laser diode unit 32 may emit light at a wavelength of 904 nanometers, or another wavelength or wavelength range, and laser diode unit 33 may emit a visible wavelength or wavelength range. A laser diode unit includes the laser diode along with additional packaging, circuitry, and power connections to function in providing laser emission.

The controller described in Gerlitz may be included in apparatus 28 so laser units 32 and 33 alternate providing light. A potential energy source 48 powers laser units 32 and 33 during standalone operation. A collimating lens 36 receives a diverging laser emission from laser diode unit 32, collimates it, and provides a collimated beam to a dichroic combiner 40. Dichroic combiner 40 allows light from two different sources to proceed along the same light path to optical comb 30. Accordingly, a collimating lens 38 receives a diverging laser emission from laser diode unit 34, collimates it, and provides a collimated beam to dichroic combiner 40.

Light passing through dichroic combiner 40 from collimating lens 36 and light reflected by dichroic combiner 40 from collimating lens 38 proceeds along the same light path to a divergence lens 42. Divergence lens 42 expands a cross-section of the laser beam received from dichroic combiner 40, thereby forming an expanding laser emission. For example, the beam may be expanded to approximately 30 millimeters in diameter. Front lens 44 is a collimating lens configured to collimate the expanding laser emission. Consequently, a dichroic combiner smaller than the surface area to be illuminated may be used in apparatus 28 and still provide a collimated laser beam with a sufficiently wide cross-section to optical comb 30.

The collimated beam may enter a dispersing diffuser (not shown), such as a 60° dispersing diffuser. Instead, the collimated beam may enter a 10° dispersing diffuser (not shown) of optical comb 30. In a “flashlight” mode without optical comb 30 attached, the 60° dispersed beam is intended to reduce the power density (in the retina) so it will be below the eye safety limit. The incoming beam into the diffuser should be about parallel (collimated) for the diffuser to emit at about 60° or 10°.

A safety mechanism may be installed to disable flashlight mode so that laser diode units 32 and 33 will not emit unless comb 30 is attached. In that case, the 10° diffuser is used before the beam enters the optical comb. The eye safety is achieved by the combination of the 10° diffuser and the geometry of light guides 34. When the 10° diffuser is included with optical comb 30, the diameter and location of front lens 44 may be altered so the emission from divergence lens 42 expands sufficiently before being collimated in front lens 44 and dispersed in the 10° diffuser to irradiate all of light guides 34.

Light therapy apparatus 28 is designed for operation with both low and high power laser diode units in the range from 1 milliWatt to 25 Watts. If follows that apparatus 28 may be a Class 1 laser device as defined according to power and wavelength by 21 CFR Part 1040 in the USA and IEC 60825 internationally. Even so, apparatus 28 may be configured as a non-Class 1 device with appropriate selection of laser diode(s). Optical combs herein that are detachable might be added to existing devices that are not Class 1 eye safe to render them eye safe with the optical comb.

FIGS. 1-3 show one example of an optical comb 10 in isometric, top, and bottom views. Optical comb 10 may be used as optical comb 30 in apparatus 28 of FIG. 3 or in other light therapy apparatuses, such as the LLLT of Gerlitz. Optical comb 10 includes a platform 12 and an array of light guides 14 extending from platform 12. Platform 10 would not necessarily provide a part of housing 46 in apparatus 28 or the Gerlitz LLLT, but it may in other applications, such as those designed not to operate with optical comb 10 detached.

Light guides 14 have respective exit surfaces 16, which would be outside a housing when optical comb 10 is coupled to a light therapy apparatus. Longitudinal axes 22 of respective light guides 14 are shown parallel to each other in FIG. 1. FIGS. 1-3 show a base 18 for each light guide 14 where it meets platform 12. FIGS. 1-3 show bases 18 as hexagonal and exit surfaces 16 as circular. Exit surfaces 16 may have other geometric shapes, such as oval.

Light guides 14 have tapered profiles 20 with cross-sections decreasing in area toward exit surfaces 16. Tapered profiles 20 allow light gathering from an input surface 24 with a wider cross-section and light delivery to a treatment surface through exit surface 16 with a narrower cross-section. An increasing gap between light guides 14 toward exit surfaces 16 allows fur or hair between light guides 14 while delivering more light source power to a treatment surface.

Exit surfaces 16 may be sized with a cross-section sufficiently small to direct light effectively into tissue between the bases of hair or fur shafts where they extend from follicles in tissue. Larger exit surfaces may have equally sized gaps between them and direct light past much of the bulk hair or fur, but still press down on the bases of hair or fur shafts so some hair or fur still exists between the exit surfaces and the tissue. Smaller exit surfaces may reduce interference by more effectively slipping between the bases of hair or fur shafts.

The bottom view in FIG. 3 shows input surfaces 24 distributed across an interior surface of platform 12. Input surfaces 24 are coplanar with platform 12 in FIG. 3, but could be curved to change their light gathering properties, if desired. Dashed lines evidence the positions of bases 18 where they meet the exterior surface of platform 12. While reference to bases 18 gives a general idea for the location of boundaries for input surfaces 24, FIG. 3 does not define the boundaries. The boundary of input surface 24 for any light guide 14 is determined by the surface area through which incident light ultimately enters light guide 14. Diffused light enters input surfaces 24 at an angle and from various directions. Accordingly, boundaries for input surfaces 24 may extend beyond the delineation for bases 18 and overlap, depending on the structure of platform 12 and the angle and direction of incident light. For collimated light incident perpendicularly to input surfaces 24, boundaries for input surfaces 24 may approximately match the delineation for bases 18.

When platform 12 and light guides 14 are a single, integral device formed from a continuous material in common, such as by a single mold, no refractive or reflective interfaces exist within platform 12 to define therein the portion functioning as light guides 14. The portion of the material in platform 12 through which light passes functions both as part of platform 12 and as part of light guides 14. Light enters input surfaces 24 on the interior surface of platform 12, where the interior surface is also part of light guides 14. The entering light passes through the dual platform 12/light guide 14 material and then enters individually defined light guides 14 at bases 18. When light guides 14 are formed separately and inserted into openings (not shown) in platform 12 to assemble optical comb 10, input surfaces 24 are well defined at the interface with platform 12. In such case, light does not pass through a dual platform 12/light guide 14 material.

Optical comb 10 includes a socket 25 (FIG. 3) for coupling with a light therapy apparatus. Socket 25 is formed by a collar 26 connected to or integrally formed with platform 12 and includes ridges 27 located radially about the circumference of collar 26. Ridges 27 may function as grips for removing and recoupling optical comb 10. Threads may be provided on the interior surface of collar 26 for a screw attachment or another structure provided for a snap attachment. Other coupling structures that permit detachment are conceivable.

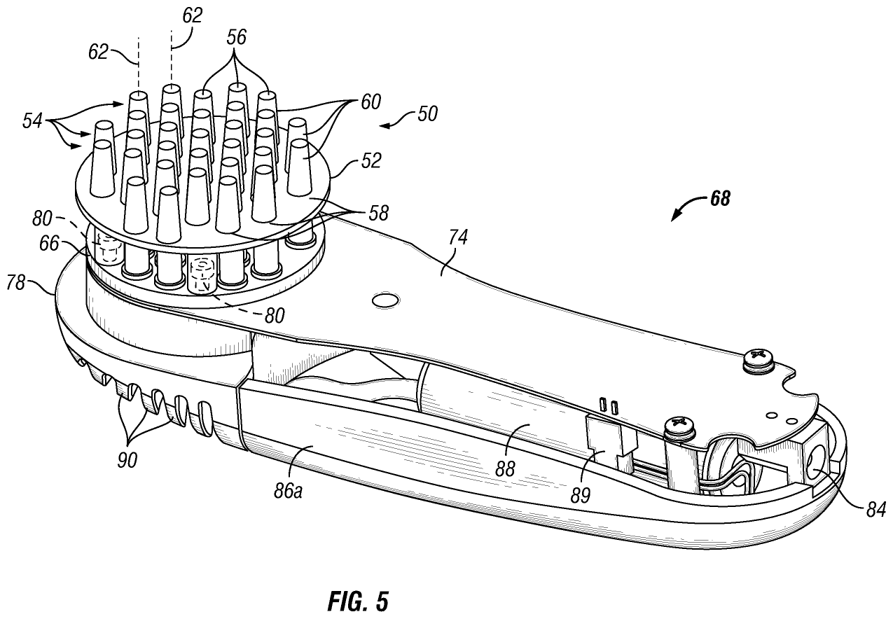

FIGS. 5-7 show views of one example of a light therapy apparatus 68. The features implemented in the FIGS. 5-7 example apparatus may also be implemented in other apparatuses herein. FIGS. 5-7 include selected components useful in understanding a method for operating apparatus 68. Additional components known to skilled persons may be included in a complete apparatus.

Apparatus 68 includes a fixed optical comb 50 different from optical comb 10 shown in FIGS. 1-3. However, it will be appreciated that features from optical comb 10 may be incorporated into optical comb 50, as an alternative, and vice versa. Optical comb 50 includes an array of light guides 54 that direct light received from a light source inside a housing 86a/b through light guides 54 to a surface outside housing 86a/b. Top housing 86a combines with bottom housing 86b to become housing 86a/b. The outside surface may be tissue targeted for treatment or another type of surface desired for light irradiation.

As light sources, apparatus 68 includes an array of LED units 72. A controller may be included in apparatus 68 to provide the irradiation features described herein. LED units 72 may emit light at a wavelength of 904 nanometers, or another wavelength or wavelength range. A potential energy source 88 powers LED units 72 during standalone operation. A lens shape 76 formed integrally with LED unit 72 directs light toward input surfaces 64 of optical comb 50. Consequently, LED units 72 directly irradiate input surfaces 64. LED units 72 are inserted into a light guide seat 66 and mounted on a printed circuit board (PCB) 74. Light guide seat 66 aligns the array of LED units 72 with the array of respective light guides 54 in optical comb 50.

Light guide seat 66 includes light wells 70 accomplishing a dual function. First, light wells 70 align light guides 54 with LED units 72, light guide seat 66 providing an interface that maintains alignment throughout use. Also, a surface defining light wells 70 provides a reflective surface for directing additional light from LED units 72 into light guides 54. Light wells 70 may be lined with a reflective coating to increase reflection efficiency. A shape for light wells 70 may be selected using optical simulation software to achieve a beneficial shape for light reflection considering the locations, geometry, and material properties of components in LED units 72, light wells 70, and light guides 54.

Light therapy apparatus 68 additionally includes user controls 82 enabling selection of settings for light emission and possibly other settings. A power socket 84 connects potential energy source 88 for charging and a cable 89 provides power to PCB 74.

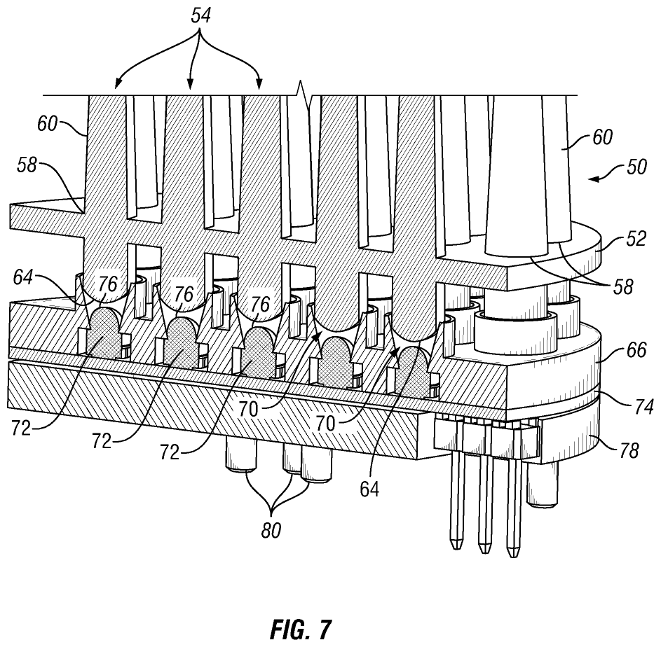

Given the array of LED units 72 generating heat during operation, a heat capacitor 78 accumulates heat from LED units 72 and dissipates heat via the increased surface area of its fins 90. Heat accumulation and dissipation passively assists with keeping temperature buildup with limits. Fasteners 80 join optical comb 50, light guide seat 66, PCB 74, and heat capacitor 78. In FIG. 7, a portion of heat capacitor 78 is cut away to view fasteners 80.

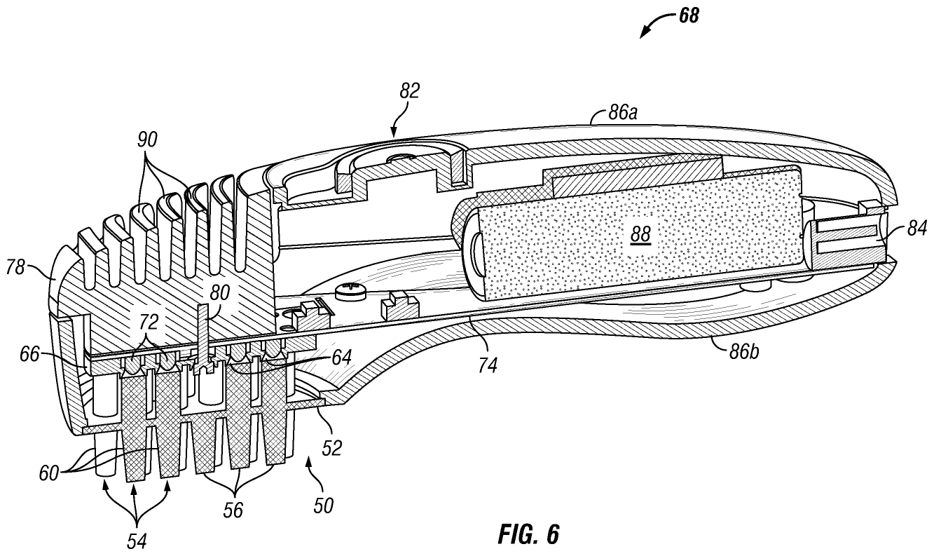

FIGS. 5-7 show one example of optical comb 50 with an array of light guides 54 extending from the interior surface of a platform 52 and input surfaces 64 convexly curved in a manner configured to focus collected light into the light guide. Optical comb 50 might additionally be used as optical comb 30 in apparatus 28 of FIG. 3 or in other light therapy apparatuses, such as the LLLT of Gerlitz. Optical comb 50 includes a platform 52 and light guides 54 extending from platform 52. Platform 50 provides a part of housing 86a/b in apparatus 68. Light guides 54 have respective exit surfaces 56, which are outside housing 86a/b when optical comb 50 is coupled to light therapy apparatus 68. Longitudinal axes 62 of respective light guides 54 are shown parallel to each other in FIG. 5. FIGS. 5 and 7 show a base 58 for each light guide 54 where it meets platform 52. FIGS. 5-6 show bases 58 as circular and exit surfaces 56 as circular. Bases 58 and exit surfaces 56 may have other geometric shapes.

Light guides 54 have tapered profiles 60 with cross-sections decreasing in area toward exit surfaces 56. Tapered profiles 60 allow light gathering from an input surface 64 with a wider cross-section and light delivery to a treatment surface through exit surface 56 with a narrower cross-section. An increasing gap between light guides 54 toward exit surfaces 56 allows fur or hair between light guides 54 while delivering more light source power to a treatment surface. Exit surfaces 56 may be sized with a cross-section sufficiently small to direct light effectively into tissue between the bases of hair or fur shafts where they extend from follicles in tissue.

When platform 52 and light guides 54 are a single, integral device formed from a continuous material in common, such as by a single mold, no refractive or reflective interfaces exist within platform 52 to define therein the portion functioning as light guides 54. The portion of the material in platform 52 through which light passes functions both as part of platform 52 and as part of light guides 54, as is apparent from cross-sections in FIGS. 6 and 7. Light enters input surfaces 64, continues through the extension of light guides 54 from the interior surface of platform 52, and passes through the dual platform 52/light guide 54 material. Light then enters light guides 54 at bases 58, continuing through the extension from the exterior surface of platform 52. When light guides 54 are formed separately and inserted into openings (not shown) in platform 52 to assemble optical comb 50, light guides 54 are well defined at the interface with platform 52. In such case, light does not pass through a dual platform 52/light guide 54 material.

Comparing optical comb 10 with optical comb 50, several contrasting features may be noted. First, optical comb 10 is detachable while optical comb 50 is fixed. Second, no gaps exist between bases 18 while gaps exist between bases 58. Third, bases 18 are hexagonal while bases 58 are circular. Fourth, input surfaces 24 are planar while input surfaces 64 are curved. Fifth, input surfaces 24 are coplanar with platform 12 while input surfaces 64 extend from platform 52. Despite selection of the five features for optical comb 10, it will be appreciated that any corresponding feature of optical comb 50 may be substituted. Likewise, despite selection of the five features for optical comb 50, it will be appreciated that any corresponding feature of optical comb 10 may be substituted.

The apparatuses and methods herein enable a variety of treatment options. Generally, optical combs herein may be used either in contact with treatment surfaces or offset some distance from treatment surfaces. For the apparatuses herein designed to be eye safe without the optical comb, the optical comb could be removed for treatment of surfaces without fur, hair, or other media to be traversed for light delivery. Especially for fixed optical combs herein, an attachment may be added to create a flat surface that transmits and distributes light for treatment of surfaces without fur, hair, or other media to be traversed for light delivery. For either detachable or fixed optical combs herein, an attachment may be added to act as a spacer. The spacer rests against a surface adjacent the treatment surface while maintaining exit surfaces at a desired distance offset from the treatment surface. In this manner, contact with open wounds could be avoided.

According to one light delivery apparatus, the apparatus includes a housing, an optical comb coupled to the housing, and at least one light source inside the housing. The housing has a size configured for the housing to be handheld. The optical comb includes a platform and an array of light guides including four or more solid light guides extending from the platform. Each of the four or more light guides contains a material that transmits light. The optical comb includes an input surface, an exit surface, and a longitudinal axis of each of the four or more light guides. The respective four or more input surfaces are inside the housing. The respective four or more exit surfaces are outside the housing. The respective four or more longitudinal axes are substantially parallel. As the term is used herein, “substantially” parallel allows for de minim is departures from perfectly parallel, such as when light guides depart slightly from parallel, but the departure has no noticeable effect on function of the light delivery apparatus.

Substantially all light emitted by the light guides exits from the exit surfaces when in operation as provided by a reflective coating on each of the four or more light guides, as provided by a geometric shape of each of the four or more light guides, or both. As the term is used herein, “substantially all” light allows for de minimis exit of light from surfaces other than the exit surface, but the lost light is insufficient to perform light therapy. The at least one light source is positioned to irradiate the input surfaces when the at least one light source is energized. Also, the at least one light source includes at least one light-emitting diode (LED) unit inside the housing or at least one laser diode inside the housing.

Additional features may be implemented in the present apparatus. By way of example, the array of four or more light guides may include 10 to 200 light guides. The platform may provide a part of the housing, functioning to enclose the internal components. A controller and a potential energy source may be inside the housing. The potential energy source, such as a battery or capacitor, permits standalone operation of the light guide without tethering to an external power source or an external light source. The controller allows users to turn on the light source and may additionally permit actuation of other control options such as power level to the light source, modulation of power over time, treatment time, selection of a light source, etc.

The optical comb may further include a tapered profile of each of the four or more light guides. The respective four or more tapered profiles are outside the housing and extend at least partially along respective exterior lengths of the four or more light guides measured from the platform to the exit surfaces. The tapered profiles may extend fully along the respective exterior lengths. As a result of the tapered profiles, a decreasing cross-section may exist along at least part of the exterior length of each of the four or more light guides, the respective four or more decreasing cross-sections decreasing in area toward the exit surfaces. Also, as a result of the tapered profiles, an increasing gap may exist between at least part of the exterior length of each of the four or more light guides, the respective four or more increasing gaps increasing in width toward the exit surfaces.

It may be that substantially no light emitted by the light guides exits from the tapered profiles when in operation due to their reflective coating, their geometric shape, or both. As the term is used herein, “substantially no” light allows for de minimis exit of light from the tapered profiles, but the lost light is insufficient to perform light therapy.

The base of a light guide, where it meets the platform, may have a variety of geometric shapes, such as hexagonal (see FIG. 2), circular (see FIG. 7), square, and others. Some shapes (e.g., hexagonal and square) allow fitting together the light guide bases so no gaps exist between them. Other shapes (e.g., circular) preclude fitting together the light guide bases and leave gaps. The presence of gaps may leave the input surfaces more conducive to irradiation with individual light sources for each light guide, reducing light loss from irradiation of gaps between bases. The reflective coating may also reduce light loss. Examples of possible coatings include silver-based and nickel-based coatings.

The platform and each of the four or more light guides may be a single, integral device formed from a continuous material in common, such as by molding, as the case may be in large quantity production. Examples of possible materials include optically transparent grades of acrylic polymers, such as poly(methyl methacrylate) (PMMA), and polycarbonate polymers. Selection of a material for a light guide may be related to a wavelength or range of wavelengths selected for the light source since different materials may transmit certain wavelengths with different efficiencies. Instead of an integral device, light guides could be formed separately and inserted into openings in a platform to assemble the optical comb, as the case may be in small quantity production.

The platform may have an exterior surface outside the housing and the light guides may extend from the exterior surface. An interior surface of the platform may be inside the housing and each of the four or more input surfaces may be coplanar with the interior surface. Instead, the light guides may extend also from the interior surface. In such case, each of the four or more input surfaces may be convexly curved in a manner configured to focus collected light into the light guide. The results of optical simulation software indicated more light could be collected by convexly curved input surfaces.

The exit surface may be substantially planar for substantially all light emitted by each of the four or more light guides. Exterior lengths of the light guides measured from the platform to the exit surfaces may range from 0.8 to 5 centimeters. Respective exterior lengths may be substantially the same for each of the four or more light guides. Surface areas for the exit surfaces may range from 0.25 to 1 square centimeter. Respective surface areas may be substantially the same for the exit surface of each of the four or more light guides. As the terms are used herein, “substantially planar” and “substantially the same” for length and area allow for de minimis differences in measurements that create no noticeable effect on function of the light delivery apparatus.

The at least one light source may include four or more LED units, each being positioned to irradiate directly a respective one of the four or more input surfaces. An LED unit includes the diode along with additional packaging, circuitry, and power connections to function in providing light emission. LED units are known often to include the diode encapsulated with a material in the shape of a lens to channel emitted light in a selected direction or in a selected cross-sectional shape. Other configurations are possible for an LED unit with or without a lens shape. However, inclusion of a lens shape or directional-type of structure in the LED unit may enhance irradiation of the input surfaces of the light guides. For direct irradiation, nothing is between the LED unit, with its lens shape, if any, and the light guide input surface. A LED unit is an example of a source of incoherent light. Other incoherent light sources may be used.

Instead, the at least one light source may include at least one laser diode and the apparatus further includes an optical system configured to collimate light from the at least one laser diode before reaching the four or more input surfaces. Possible components of an optical system between a light source and an input surface include lenses, dichroic combiners, diffusers, etc. to achieve beneficial effects, such as discussed elsewhere herein. A laser diode is an example of a source of coherent light. Thus, light delivery apparatuses herein may use coherent or incoherent light sources.

The additional features that may be implemented in the present apparatus may also be implemented in other embodiments herein. Specifically, any of the features described for the present apparatus, alone or in combination, may be implemented in the related two apparatuses that immediately follow, unless they technically conflict.

According to another light delivery apparatus, the apparatus includes a housing, an optical comb coupled to the housing, and at least one light source inside the housing. The housing has a size configured for the housing to be handheld. The optical comb includes a platform and an array of light guides including four or more solid light guides extending from the platform. The platform provides a part of the housing. Each of the four or more light guides contains a material that transmits light. The optical comb includes an input surface, an exit surface, and a longitudinal axis of each of the four or more light guides. The respective four or more input surfaces are inside the housing. The respective four or more exit surfaces are outside the housing and are and substantially planar for substantially all light emitted by each of the four or more light guides. The respective four or more longitudinal axes are substantially parallel. An exterior surface of the platform is outside the housing, the light guides extending from the exterior surface. An interior surface of the platform is inside the housing, the light guides extending also from the interior surface and each of the four or more input surfaces being convexly curved in a manner configured to focus collected light into the light guide.

Substantially all light emitted by the light guides exits from the exit surfaces when in operation as provided by a reflective coating on each of the four or more light guides, a geometric shape of each of the four or more light guides, or both. The at least one light source is positioned to irradiate the input surfaces when the at least one light source is energized. The at least one light source includes four or more LED units inside the housing, each being positioned to irradiate directly a respective one of the four or more input surfaces.

Additional features may be implemented in the present apparatus. By way of example, the features of the immediately preceding apparatus may be implemented.

According to a further light delivery apparatus, the apparatus includes a housing, an optical comb coupled to the housing, and at least one light source inside the housing. The housing has a size configured for the housing to be handheld. The optical comb includes a platform and an array of light guides including four or more solid light guides extending from the platform. The platform provides a part of the housing. Each of the four or more light guides contains a material that transmits light. The optical comb includes an input surface, an exit surface, and a longitudinal axis of each of the four or more light guides. The respective four or more input surfaces are inside the housing. The respective four or more exit surfaces are outside the housing and are and substantially planar for substantially all light emitted by each of the four or more light guides. The respective four or more longitudinal axes are substantially parallel. An exterior surface of the platform is outside the housing, the light guides extending from the exterior surface. An interior surface of the platform is inside the housing, each of the four or more input surfaces being coplanar with the interior surface.

Substantially all light emitted by the light guides exits from the exit surfaces when in operation as provided by a reflective coating on each of the four or more light guides, a geometric shape of each of the four or more light guides, or both. The at least one light source is positioned to irradiate the input surfaces when the at least one light source is energized. The at least one light source includes at least one laser diode inside the housing. The apparatus includes an optical system configured to collimate light from the at least one laser diode before reaching the four or more input surfaces.

Additional features may be implemented in the present apparatus. By way of example, the features of the immediately preceding two apparatuses may be implemented.

The inventors expressly contemplate that the various options described herein for individual methods and apparatuses are not intended to be so limited except where incompatible. The features and benefits of individual methods herein may also be used in combination with apparatuses and other methods described herein even though not specifically indicated elsewhere. Similarly, the features and benefits of individual apparatuses herein may also be used in combination with methods and other apparatuses described herein even though not specifically indicated elsewhere.

In compliance with the statute, the embodiments have been described in language more or less specific as to structural and methodical features. It is to be understood, however, that the embodiments are not limited to the specific features shown and described. The embodiments are, therefore, claimed in any of their forms or modifications within the proper scope of the appended claims appropriately interpreted in accordance with the doctrine of equivalents.

TABLE OF REFERENCE NUMERALS FOR FIGS.

| | |
|---|---|
| 10 | optical comb |
| 12 | platform |
| 14 | light guide |
| 16 | exit surface |
| 18 | base |
| 20 | tapered profile |
| 22 | longitudinal axis |
| 24 | input surface |
| 25 | socket |
| 26 | collar |
| 27 | ridge |
| 28 | light therapy apparatus |
| 30 | optical comb |
| 32 | laser diode unit |
| 33 | laser diode unit |
| 34 | light guide |
| 36 | collimating lens |
| 38 | collimating lens |
| 40 | dichroic combiner |
| 42 | divergence lens |
| 44 | front lens |
| 46 | housing |
| 48 | potential energy source |
| 50 | optical comb |
| 52 | platform |
| 54 | light guide |
| 56 | exit surface |
| 58 | base |
| 60 | tapered profile |
| 62 | longitudinal axis |
| 64 | input surface |
| 66 | light guide seat |
| 68 | light therapy apparatus |
| 70 | light well |
| 72 | LED unit |
| 74 | PCB |
| 76 | lens shape |
| 78 | heat capacitor |
| 80 | fastener |
| 82 | user controls |
| 84 | power socket |
| 86a | top housing |
| 86b | bottom housing |
| 88 | potential energy source |
| 89 | cable |
| 90 | fins |

What is claimed is:

1. A light delivery apparatus comprising:
a housing having a size configured for the housing to be handheld;
an optical comb coupled to the housing and including:
a platform;
an array of light guides including four or more solid light guides extending from the platform, each of the four or more light guides containing a material that transmits light;
an input surface of each of the four or more light guides, the respective four or more input surfaces being inside the housing;
an exit surface of each of the four or more light guides, the respective four or more exit surfaces being outside the housing;
a longitudinal axis of each of the four or more light guides, the respective four or more longitudinal axes being substantially parallel; and
a reflective coating on each of the four or more light guides, a geometric shape of each of the four or more light guides, or both that is configured so substantially all light emitted by the light guides exits from the exit surfaces when in operation; and
at least one light source inside the housing and positioned to irradiate the input surfaces when the at least one light source is energized, the at least one light source including at least one light-emitting diode (LED) unit inside the housing or at least one laser diode inside the housing.

2. The apparatus of claim 1 wherein the platform provides a part of the housing.

3. The apparatus of claim 1 wherein the optical comb further comprises:
a tapered profile of each of the four or more light guides, the respective four or more tapered profiles being outside the housing and extending at least partially along respective exterior lengths of the four or more light guides measured from the platform to the exit surfaces;
a decreasing cross-section along at least part of the exterior length of each of the four or more light guides, the respective four or more decreasing cross-sections decreasing in area toward the exit surfaces as a result of the tapered profiles;
an increasing gap between at least part of the exterior length of each of the four or more light guides, the respective four or more increasing gaps increasing in width toward the exit surfaces as a result of the tapered profiles; and
the reflective coating of each of the four or more light guides, the geometric shape of each of the four or more light guides, or both being configured so that substantially no light emitted by the light guides exits from the tapered profiles when in operation.

4. The apparatus of claim 3 wherein the tapered profiles extend fully along the respective exterior lengths.

5. The apparatus of claim 1 wherein the platform and each of the four or more light guides are a single, integral device formed from a continuous material in common.

6. The apparatus of claim 1 wherein an exterior surface of the platform is outside the housing and the light guides extend from the exterior surface.

7. The apparatus of claim 6 wherein an interior surface of the platform is inside the housing and each of the four or more input surfaces are coplanar with the interior surface.

8. The apparatus of claim 6 wherein an interior surface of the platform is inside the housing, the light guides extend also from the interior surface, and each of the four or more input surfaces are convexly curved in a manner configured to focus collected light into the light guide.

9. The apparatus of claim 1 wherein the exit surface is substantially planar for substantially all light emitted by each of the four or more light guides.

10. The apparatus of claim 1 wherein:
respective exterior lengths are substantially the same for each of the four or more light guides measured from the platform to the exit surfaces and range from 0.8 to 5 centimeters; and
respective surface areas are substantially the same for the exit surface of each of the four or more light guides and range from 0.25 to 1 square centimeter.

11. The apparatus of claim 1 wherein the at least one light source comprises four or more LED units, each being positioned to irradiate directly a respective one of the four or more input surfaces.

12. The apparatus of claim 1 wherein the at least one light source comprises at least one laser diode and the apparatus further comprises an optical system configured to collimate light from the at least one laser diode before reaching the four or more input surfaces.

13. A light delivery apparatus comprising:
a housing having a size configured for the housing to be handheld;
an optical comb coupled to the housing and including:
a platform that provides a part of the housing;
an array of light guides including four or more solid light guides extending from the platform, each of the four or more light guides containing a material that transmits light;
an input surface of each of the four or more light guides, the respective four or more input surfaces being inside the housing;
an exit surface of each of the four or more light guides, the respective four or more exit surfaces being outside the housing and substantially planar for substantially all light emitted by each of the four or more light guides;
an exterior surface of the platform outside the housing, the light guides extending from the exterior surface;
an interior surface of the platform inside the housing, the light guides extending also from the interior surface and each of the four or more input surfaces being convexly curved in a manner configured to focus collected light into the light guide;
a longitudinal axis of each of the four or more light guides, the respective four or more longitudinal axes being substantially parallel; and
a reflective coating on each of the four or more light guides, a geometric shape of each of the four or more light guides, or both that is configured so substantially all light emitted by the light guides exits from the exit surfaces when in operation; and
at least one light source inside the housing and positioned to irradiate the input surfaces when the at least one light source is energized, the at least one light source including four or more LED units inside the housing, each being positioned to irradiate directly a respective one of the four or more input surfaces.

14. The apparatus of claim 13 wherein the optical comb further comprises:
a tapered profile of each of the four or more light guides, the respective four or more tapered profiles being outside the housing and extending at least partially along respective exterior lengths of the four or more light guides measured from the platform to the exit surfaces;
a decreasing cross-section along at least part of the exterior length of each of the four or more light guides, the respective four or more decreasing cross-sections decreasing in area toward the exit surfaces as a result of the tapered profiles;
an increasing gap between at least part of the exterior length of each of the four or more light guides, the respective four or more increasing gaps increasing in width toward the exit surfaces as a result of the tapered profiles; and
the reflective coating of each of the four or more light guides, the geometric shape of each of the four or more light guides, or both being configured so that substantially no light emitted by the light guides exits from the tapered profiles when in operation.

15. The apparatus of claim 14 wherein the tapered profiles extend fully along the respective exterior lengths.

16. The apparatus of claim 15 wherein the platform and each of the four or more light guides are a single, integral device formed from a continuous material in common.

17. A light delivery apparatus comprising:
a housing having a size configured for the housing to be handheld;
an optical comb coupled to the housing and including:
a platform that provides a part of the housing;
an array of light guides including four or more solid light guides extending from the platform, each of the four or more light guides containing a material that transmits light;
an input surface of each of the four or more light guides, the respective four or more input surfaces being inside the housing;
an exit surface of each of the four or more light guides, the respective four or more exit surfaces being outside the housing and substantially planar for substantially all light emitted by each of the four or more light guides;
an exterior surface of the platform outside the housing, the light guides extending from the exterior surface;
an interior surface of the platform inside the housing, each of the four or more input surfaces being coplanar with the interior surface;
a longitudinal axis of each of the four or more light guides, the respective four or more longitudinal axes being substantially parallel; and
a reflective coating on each of the four or more light guides, a geometric shape of each of the four or more light guides, or both that is configured so substantially all light emitted by the light guides exits from the exit surfaces when in operation;
at least one light source inside the housing and positioned to irradiate the input surfaces when the at least one light source is energized, the at least one light source including at least one laser diode inside the housing; and
an optical system configured to collimate light from the at least one laser diode before reaching the four or more input surfaces.

18. The apparatus of claim 17 wherein the optical comb further comprises:
a tapered profile of each of the four or more light guides, the respective four or more tapered profiles being outside the housing and extending at least partially along respective exterior lengths of the four or more light guides measured from the platform to the exit surfaces;
a decreasing cross-section along at least part of the exterior length of each of the four or more light guides, the respective four or more decreasing cross-sections decreasing in area toward the exit surfaces as a result of the tapered profiles;
an increasing gap between at least part of the exterior length of each of the four or more light guides, the respective four or more increasing gaps increasing in width toward the exit surfaces as a result of the tapered profiles; and
the reflective coating of each of the four or more light guides, the geometric shape of each of the four or more light guides, or both being configured so that substantially no light emitted by the light guides exits from the tapered profiles when in operation.

19. The apparatus of claim 18 wherein the tapered profiles extend fully along the respective exterior lengths.

20. The apparatus of claim 19 wherein the platform and each of the four or more light guides are a single, integral device formed from a continuous material in common.
